(19) United States
(12) Patent Application Publication
(10) Pub. No.: US 2014/0302453 A1
(43) Pub. Date: Oct. 9, 2014

(54) ENDOSCOPE WITH AT LEAST ONE OPTICAL SENSOR ARRANGEMENT AND METHOD FOR PRODUCING AND USING SAME

(71) Applicant: AWAIBA HOLDING S.A., Yverdon-les-Bains (CH)

(72) Inventor: Martin WANY, Yverdon-les-Bains (CH)

(21) Appl. No.: 14/311,903

(22) Filed: Jun. 23, 2014

Related U.S. Application Data
(63) Continuation of application No. PCT/CH2012/000279, filed on Dec. 21, 2012.

(30) Foreign Application Priority Data
Dec. 23, 2011 (CH) 2046/11

(51) Int. Cl. A61B 1/00; A61B 1/24

(52) U.S. Cl. CPC A61B 1/00124; A61B 1/24; A61B 1/0011
USPC 433/29; 600/160; 29/874

(57) ABSTRACT

A method for producing an endoscope with an optical sensor arrangement with a distal side and a proximal side and with an electrical connection between the distal side and the proximal side. The optical sensor arrangement is mounted directly on the electrical conductor, The main feature of the invention relates to the fact that the electrical contacts, on the optical sensor, are designed so that they are arranged in a single row and that the contacts (11, 21), in this single row, are arranged with a period that corresponds to the period of the single wires found in a contact area of the electrical connection.

This application is a continuation of PCT/CH2012/000279 filed Dec. 21, 2012 which claims priority from Swiss Application Serial No. 2046/11 filed Dec. 23, 2011.

### FIELD OF THE INVENTION

The invention relates to an endoscope with an optical sensor arrangement with a distal side and a proximal side and an electrical connection between the distal side and the proximal side, wherein the electrical connection consists of a plurality of single wires and wherein the at least one optical sensor is mounted directly on the electrical connecting cable between the distal and the proximal side, without intermediate connection on a printed, photolithographically structured or pressed electrical circuit board.

It also relates to a method for producing an endoscope with an optical sensor arrangement with electrical conductors, wherein the electrical conductors are prepared in a first phase and the image sensor or camera module is applied on the prepared conductors in a second phase.

### BACKGROUND OF THE INVENTION

Endoscopes are often used for medical examinations, interventions and analyses. In order to bring the endoscope to the examination and operation site with the most limited interventions possible for the patient, one tries to use an endoscope with the smallest diameter possible. On the other hand, efforts are made to increase the location resolution of the endoscopic camera. Efforts are made also to produce increasingly disposable endoscopes, which exclude the risks and costs of sterilization. However, to allow disposable endoscopes, the design of their structure must be simple and cost-effective.

The progress in the integration of miniaturized CMOS image sensors allows producing increasingly endoscopes that already integrate the CMOS image sensor in the tip of the endoscope. Refer in particular to the article “Miniature Form Factor digital-image sensor for endoscopic applications” by M. Wany et. al. published in the magazine: SPIE Photonics West in January 2009 under reference E109-E1114-9 7249-32. In order to reduce the size of the image sensors and to allow a compact construction of the sensors and optic in the endoscope tip, so-called “Chip Scale Packaging” technologies, in particular “Through Silicon Via Package” technologies, can be used. These technologies allow limiting the dimensions of the image sensor construction to the size of the sensor chip and reducing production costs, in particular for small sensors. But, usually, these construction technologies have a two-dimensional connection points matrix, the so-called “Ball Grid Array”, which must be applied for electrical connection on a circuit board, which in turn must be connected to the electrical conductors. This construction type however requires a relatively large installation space and leads to relatively high expenses, so that the commercial spreading of this kind of image sensors did not develop as desired for endoscopes.

The American patent application US 2008/0091064 A1 relates to a miniaturized endoscope arrangement that can be produced at a low price and operated with low energy consumption. But this arrangement does not use the production technology of the invention, the various partial modules are connected together by means of cables. FIG. 2 shows that the image sensor has a BGA Array (according to the prior art a two-dimensional array) and is to be mounted on a flexible circuit board. Moreover, the image sensor is described as a “micro ball grid array”, that is to say a two-dimensional array, and is mounted on a “flexible circuit board”, and not directly on wires. The image sensor used is explicitly a standard image sensor as used in the mobile phone technology, where two-dimensional BGA contact arrays are used as a standard. In addition, the claim states explicitly that it means an arrangement with an image sensor that is electrically connected by means of a two-dimensional “micro ball grid array” contact matrix.

The American patent application US 2005/0285973 A1 describes an imaging camera mounted on a flexible circuit. The sensor is mounted mechanically directly on a FPS, but it is not connected electrically with it (wire bonding is required). Direct application of sensor BUMPs on a FPC (but not on a one-dimensional array or on wires). FIG. 2 describes how the sensor is mounted mechanically directly on a FPC, while the electrical connection is nevertheless to be achieved by means of wire bonding. It explains explicitly that the sensor is mounted directly, but in an electrically NON-conductive way, on a FPC, while the reduction of the components remains limited to a FPC as a substrate and a process for mounting on a FPC (but without the details of the direct electrical connection operation).

The European patent application EP 1104182 A1 relates to an image receiver that is mounted on an at least partly flexible printed circuit for an endoscope arrangement. FIG. 3 shows the cross-section of a standard BGA-type Chip Scale Package, that is to say a package with a two-dimensional contact arrangement, as visible in the following FIGS. 4 and 5. In addition, this invention describes the mounting directly on a FPC, but not on parallel wires. It does neither describe a one-dimensional contact arrangement.

Based on this prior art, the invention aims to produce an optical sensor, in particular an image sensor, characterized in that the electrical connection consists of a plurality of single conductors forming a one-dimensional network, and in that the optical sensor arrangement is mounted directly on said electrical conductors provided with an insulation, without intermediate connection on an electrical circuit board, while the period of the network corresponds to the period found in the contact area of the electrical conductors.

### SUMMARY OF THE INVENTION

The invention shows means and process techniques with which an optical sensor, advantageously an image sensor or a miniature camera module including an image sensor and a optical lens, can be mounted directly on an electrical conductor, as well as a method for preparing in a cost-effective way electrical conductors so that the optical sensor can be mounted on these thanks to the described mounting processes.

Therefore, the endoscope with an optical sensor according to the invention is characterized in that the electrical contacts on the optical sensor are designed so that they are arranged in one single row and that the contacts (11, 21) in this row are arranged with a period that corresponds to the period of the single wires found in a contact area of said electrical connection.

Said contacts are advantageously longer in the direction orthogonal to the row of contacts than they are in the direction of the row of contacts.

Said electrical conductor includes preferably a ribbon cable with at least two separated wires.

The method according to the invention is characterized in that, during the second phase, thermally hardening adhesive with electrically conductive particles and that reduces in volume when hardening is used to fasten the optical sensor arrangement onto the conductor.

Thermally hardening adhesive enriched with electrically conductive particles and that reduces in volume when hardening is used preferably.

The electrical and mechanical connection between the metals of the electrical conductor and the contacts of said image sensor or camera module is achieved preferably by creating a durable connection by means of mechanical pressure and excitation with ultrasonic energy.

Said electrical conductors or said image sensor or camera module, or both parts, are preferably plasma-activated prior to the connecting process.

During the first phase, the electrical conductor is preferably held mechanically and pressed on one of its sides against a mechanical stop, while the insulation of the conductor is removed mechanically from the other side,

Advantageously, during the first phase of removing the electrical insulation from the conductor, also a part of the electrical conductor is removed in order to achieve an at least partly flat surface on the electrical conductor.

According to a variant of this method, the electrical insulation of the conductor is removed in a contact area during the first phase by means of laser processing.

During the second phase, the electrical and mechanical connection between the metals of the electrical conductor and the contacts of said optical sensor arrangement is achieved preferably by creating a durable connection by means of mechanical pressure and excitation with ultrasonic energy.

During the second phase, the electrical and mechanical connection between the metals of the electrical conductor and the contacts of said optical sensor arrangement can be achieved by creating a durable connection by means of mechanical pressure and excitation with ultrasonic energy.

Said electrical conductors or said optical sensor, or both parts, are advantageously plasma-activated prior to the connecting process.

During the second phase, the electrical and mechanical connection between the application of the optical sensor and the electrical conductor can be achieved by means of a soldering method,

The heat supply for melting the solder is advantageously provided by an optical source.

The heat supply for melting the solder is preferably provided by the transfer of the heat from a heat source that is in mechanical contact with the electrical conductor,

### BRIEF DESCRIPTION OF THE DRAWINGS

An implementation example of the invention is described below in reference to the drawings. The drawings represent:

FIG. 1 shows an arrangement of electrical connections in a two-dimensional connection matrix (Ball Grid Array) according to the prior art,

FIG. 2 shows an arrangement of electrical connections in a one-dimensional connection matrix,

FIG. 3 shows an arrangement of electrical connections in a one- dimensional connection matrix, and

FIG. 4 shows an optical sensor arrangement that is mounted directly on an electrical conductor cable.

### DETAILED DESCRIPTION OF THE DRAWINGS

FIG. 1 shows an arrangement of electrical connections 2 on an optical sensor arrangement 1 in a two-dimensional connection matrix (Ball Grid Array) according to the prior art.

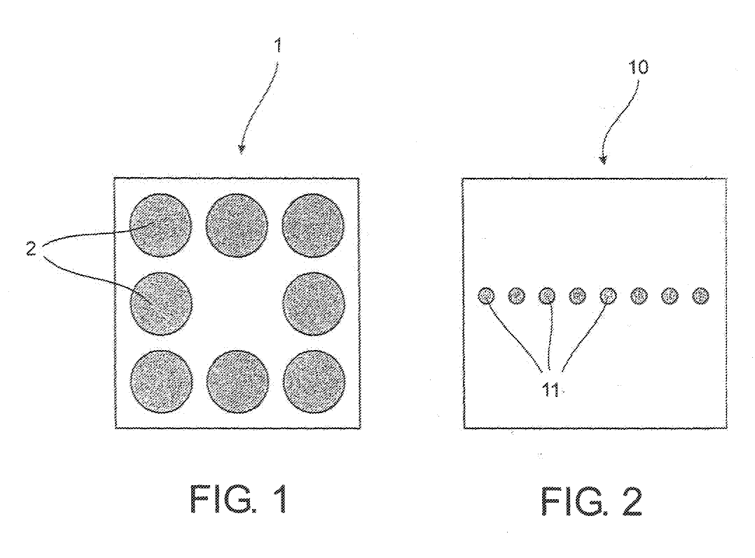

FIG. 2 shows the arrangement according to the invention of electrical connections 11 on an optical sensor arrangement 10 in a one-dimensional connection matrix. The electrical connections can be made out of gold, copper or zinc or zinc alloys, or out of other electrically conductive materials.

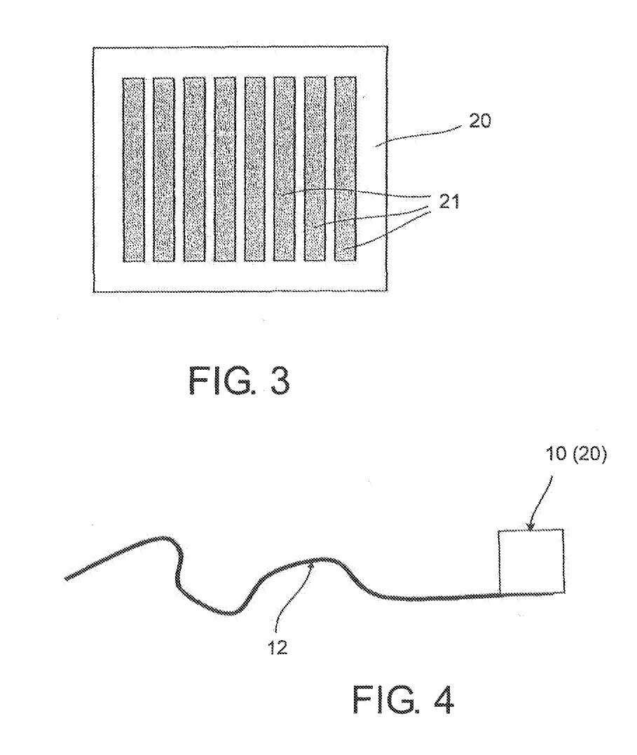

FIG. 3 shows the arrangement according to the invention of electrical connections 21 on an optical sensor arrangement 20 in a one-dimensional connection matrix in the form of a row of contacts wherein the connections 21 have a rectangular shape, so that the connections are longer than they are wide in the direction orthogonal to the row of contacts. The electrical connections can be made out of gold, copper or zinc or zinc alloys, or out of other electrically conductive materials.

FIG. 4 shows the arrangement according to the invention of an optical sensor arrangement 10 (or 20) that is mounted directly on an electrical conductor 12.

The possibilities of realizing the invention disclosed in this description or in the drawings must be understood merely as illustrative examples. The examples do not restrict the general nature of the invention. The protection of the invention is set out only in the attached patent claims.

The mounting according to the invention of optical sensor arrangements, called image sensors or respectively miniature cameras, on electrical conductors without using an electric circuit board can be achieved by applying the sensor directly on a bare surface of the electrical conductor whose conductive tracks are applied parallel in the sensor area. In order to make this possible, the contacts matrix is formed in the sensor 10 according to the invention as a one-dimensional array, a so-called row of contacts 11. The contacts are formed to be longer in the direction orthogonal to the row of contacts than in the direction of the row of contacts 21. Optionally, the contacts are formed so that the surface of the contact points protrudes from the plane of the sensor arrangement mounting surface and so that these are flat or almost flat in at least a small area.

The electrical conductor consists of a plurality of single wires, which run in parallel over the whole length of the conductor (e.g., ribbon cable), or of electrical wires that are laid in parallel in the area where the sensor is to be mounted. The electrical conductors can be produced both by means of photolithographic processes or “extrusion” processes. In the case of electrical conductors produced by extrusion, their insulation must be removed in the area where the sensor arrangement is to be mounted prior to the assembly. In the method according to the invention, this is achieved by holding the electrical conductor mechanically and pressing one of its sides against a steady rest. The electrical insulation is removed mechanically from its free side, advantageously using a rotary abrasive tool. When removing the insulation, a part of the conductor is also removed in order to remove at least a part of the round conductor surface and to create an at least partly flat contact surface on the electrical conductor. In an alternative conductor preparation method, the insulation is removed by means of a laser. Advantageously, in this case, only the insulation is removed, without processing the conductor, which keeps its original shape. The preparation of the electrical conductors to equip the cameras can optionally be designed so that the same fixture holds a plurality of electrical conductors together, in order to be able to equip the optical sensor arrangements in parallel at a later stage.

The optical sensor arrangement, in particular an image sensor or a miniature camera module, is mounted directly on the electrical conductor. In the method according to the invention, this occurs by applying between the conductor and the optical sensor arrangement a thermally hardening adhesive enriched with electrically conductive particles that reduces in volume when hardening. The optical sensor arrangement is pressed against the conductor by means of mechanical pressure while thermal energy hardens the adhesive. The quantity and size of the electrically conductive particles is to be selected so that, thanks to the mechanical pressure applied during the hardening, particles are trapped between the respective electrical conductors and the corresponding contacts on the side of the optical sensor arrangement, creating an ohmic contact between the optical sensor arrangement and the corresponding wire, but without electrical contact with the adjacent wires.

In an alternative embodiment of the construction method according to the invention, the sensor is applied directly on the electrical conductor without joining products. A durable electrical and mechanical assembly is achieved by creating a metallic connection between the contacts of the optical sensor arrangement and the electrical wires thanks to mechanical pressure and ultrasonic energy. To improve the quality of the connection, the optical sensor arrangement and/or the electrical wires can optionally be cleaned and activated by a plasma process.

In another alternative embodiment of the construction method according to the invention, the sensor is applied directly on the electrical conductor by means of a soldering method.

The use of the image sensor mounted according to the invention directly on an electrical cable is especially interesting for applications in medical and industrial endoscope arrangements, as the optical sensor arrangement has minimal dimensions, which allows the production of small-diameter endoscopes. Furthermore, the mounting of the sensors, respectively of the camera modules, directly on a cable, without electrical circuit board, allows costs reduction, which allows the production of disposable devices.

1. An optical sensor arrangement for use in particular in an endoscope with a distal side and a proximal side and an electrical connection between the distal side and the proximal side,
wherein the electrical connection comprises a plurality of single wires, and
the optical sensor is mounted directly on the electrical connecting cable between the distal and the proximal side, without intermediate connection on a printed, photolithographically structured or pressed electrical circuit board.

2. The optical sensor arrangement according to claim 1, wherein the electrical contacts on the optical sensor are designed so that the electrical contacts are arranged in one single row and the contacts (11, 21) in the single row are arranged with a period that corresponds to the period of the single wires found in a contact area of the electrical connection.

3. The optical sensor arrangement according to claim 2, wherein the contacts (21) are longer in a direction orthogonal to the row of contacts than in a direction of the row of contacts.

4. The optical sensor arrangement according to claim 1, wherein the electrical conductor consists of a ribbon cable with at least two separated electrical wires.

5. A method of producing an optical sensor arrangement with an electrical conductor for use in an endoscope with a distal side and a proximal side and an electrical connection between the distal side and the proximal side, wherein the electrical connection comprises a plurality of single wires, and the optical sensor is mounted directly on the electrical connecting cable between the distal and the proximal side, without intermediate connection on a printed, photolithographically structured or pressed electrical circuit board, the method comprising the steps of:
preparing the electrical conductors during a first phase, and
applying the image sensor or camera module on the prepared conductors during a second phase.

6. The method of producing an optical sensor arrangement according to claim 5, further comprising the step of, during the second phase, thermally hardening adhesive enriched with electrically conductive particles and that reduces in volume when hardening is used to fix the optical sensor arrangement on the conductor.

7. The method of producing an optical sensor arrangement according to claim 5, further comprising the step of, during the second phase, achieving the electrical and mechanical connection between the metals of the electrical conductor and the contacts of the optical sensor arrangement by creating a durable connection by mechanical pressure and excitation with ultrasonic energy.

8. The method of producing an optical sensor arrangement according to claim 5, further comprising the step of plasma-activating at least one of the electrical conductor and the image sensor or camera module prior to the connecting process.

9. The method of producing an optical sensor arrangement according to claim 5, further comprising the step of, during the second phase, achieving the electrical and mechanical connection, between the optical sensor arrangement and the electrical conductor, by a soldering method.

10. The method of producing an optical sensor arrangement according to claim 9, further comprising the step of supplying the heat supply, for melting the solder, by an optical source.

11. The method of producing an optical sensor arrangement according to claim 9, further comprising the step of supplying the heat supply, for melting the solder, by transfer the heat from a heat source that is in mechanical contact with the electrical conductor.

12. The method of producing an optical sensor arrangement according to claim 9, further comprising the step of providing the heat supply, for melting the solder, by a jet of hot gas.

13. The application of the optical sensor arrangement according to claim 1, further comprising the step of mounting the optical sensor arrangement in an endoscope.

14. The application of the optical sensor arrangement according to claim 1, further comprising the step of using the optical sensor arrangement in a human body.

15. The application of the optical sensor arrangement according to claim 1, further comprising the step of using the optical sensor arrangement in a field of dentistry for imaging examination.
